(19) United States
(12) Patent Application Publication
(10) Pub. No.: US 2007/0092575 A1
(43) Pub. Date: Apr. 26, 2007

(54) Compositions for administering RNAIII-inhibiting peptides

(76) Inventors: Naomi Balaban, Hopkinton, MA (US); Joel Braunstein, Pikesville, MD (US)

Correspondence Address: MERCHANT & GOULD PC, P.O. BOX 2903, MINNEAPOLIS, MN, 55402-0903, US

(21) Appl. No.: 11/430,091

(22) Filed: May 9, 2006

Related U.S. Application Data
(60) Provisional application No. 60/679,516, filed on May 10, 2005.

(51) Int. Cl. A61K 9/14; A61K 38/10; A61K 38/08

(52) U.S. Cl. 424/489; 514/12; 514/17; 514/16; 977/906

(57) ABSTRACT

Compositions comprising RIP are advantageously formulated in compositions allowing sustained release and protection from degradation, and improved therapeutic efficacy. To that end, RIP compositions may be delivered to the skin or mucosal membranes as a salve or the like. Alternatively or additionally, RIP compositions may be administered in polymeric nanoparticle carriers, which may be biodegradable. Such formulations are compatible with oral administration. The nanoparticle may accommodate a composition comprising a RIP and at least one other antimicrobial agent, e.g., an antibiotic or an antimicrobial peptide. The nanoparticle further may comprise a coating or moiety, such as an antibody or fragment thereof, to assist in cell targeting.

### CROSS REFERENCE TO RELATED CASES

This application claims the benefit of Provisional U.S. Application Ser. No. 60/679,516, filed May 10, 2005, which is incorporated by reference herein in its entirety.

### BACKGROUND

### TECHNICAL FIELD

This application relates generally to pharmacological compositions for delivering an RNAIII-inhibiting peptide to a mucosal surface with or without a nanoparticle carrier system.

RNAIII-inhibiting Peptides

Recent studies have evidenced the importance of quorum-sensing in the pathology of bacterial species including Vibrio cholerae, Pseudomonas aeruginosa, and Staphylococcus aureus. Quorum-sensing is a mechanism through which a bacterial population receives input from neighboring cells and elicits an appropriate response to enable itself to survive within the host. See Balaban et al., Science 280: 438-40 (1998); Miller et al., Cell 110: 303-14 (2002); Hentzer et al., EMBO J. 22: 3803-15 (2003); Korem et al., FEMS Microbiol. Lett. 223: 167-75 (2003). In Staphylococcus, quorum-sensing controls the expression of proteins implicated in bacterial virulence, including colonization, dissemination, and production of multiple toxins involved in disease promotion. Some of these virulence factors are enterotoxins and toxic-shock syndrome toxin-1 (TSST-1) that act as superantigens to cause over-stimulation of the host immune system, causing excessive release of cytokines and inducing the hyper-proliferation of T cells.

In a quorum-sensing system in S. aureus, the effector quorum-sensing molecule RNAIII-activating peptide (RAP) phosphorylates “target of RNAIII-activating protein” (TRAP), a 21 kDa protein that is highly conserved among Staphylococcus. TRAP phosphorylation promotes bacterial adhesion and the downstream production of a regulatory RNA molecule termed RNAIII, which is responsible for toxin synthesis. Balaban (1998); Balaban et al., J. Biol. Chem. 276: 2658-67 (2001). An antagonist of RAP, RNAIII-inhibiting peptide (RIP), inhibits the phosphorylation of TRAP and thereby strongly inhibits the downstream production of virulence factors, bacterial adhesion, biofilm formation, and infections in vivo. The mechanism of action of RIP is different from common antibiotics: instead of killing bacteria, RIP inhibits bacterial cell-cell communication, rendering the bacteria more vulnerable to host defense mechanisms. See Balaban (1998); Balaban et al., Peptides 21: 1301-11 (2000); Gov et al., Peptides 22: 1609-20 (2001); Balaban et al., J. Infect. Dis. 187:625-30 (2003); Cirioni et al., Circulation 108: 767-71 (2003); Ribeiro et al., Peptides 24: 1829-36 (2003); Giacometti et al., Antimicrob. Agents Chemother. 47: 1979-83 (2003); Balaban et al., Kidney Int. 23: 340-45 (2003); Balaban et al., Antimicrob. Agents Chemother. 48: 2544-50 (2004); Dell'Acqua et al., J. Infect. Dis. 190: 318-20 (2004).

Colloidal Drug Carriers

Colloidal drug carriers, such as liposomes and nanoparticles, have been used to improve the therapeutic index of both established and new drug molecules by modifying their biodistribution, and thus increasing their efficacy and/or reducing their toxicity. The distribution of the encapsulated drug follows that of the carrier, rather than depending on the physicochemical properties of the drug itself. Wasan et al., Immunopharmacol. Immunotoxicol. 17: 1-15 (1995).

Liposomes are small vesicles consisting of one or more concentric lipid bilayers surrounding aqueous compartments. The rationale for using liposomes as carriers for drugs is improvement of the efficacy of the drug by modifying its pharmacokinetics and tissue distribution. Allen, Drugs 54: 8-14 (1997). Liposomes are considered to be versatile delivery systems. The liposomal size and other physicochemical characteristics, e.g., surface charge, surface coating, and bilayer rigidity, can be easily manipulated by changing the lipid composition. In this manner, the biological distribution, stability and cellular interaction of liposomes can be predictably tailored. Pinto-Alphandry et al., Int. J. Antimicrob. Agents 13: 155-68 (2000); Abra et al., Biochim. Biophys. Acta 666: 493-503 (1988). The use of liposomes as carriers for RNAIII-inhibiting peptides, for example, is described in applications Ser. No. 10/358,448, filed Feb. 3, 2003; Ser. No. 09/839,695, filed Apr. 19, 2001; and Ser. No. 09/054,331, filed Apr. 2, 1998, now U.S. Pat. No. 6,291,431, each of which is incorporated herein by reference in its entirety.

### SUMMARY

Compositions for administering an RNAIII-inhibiting peptide advantageously provide sustained, high local concentrations of RIP at a site of infection or at a site at risk of infection. The invention provides for application of RIP compositions to external surfaces of a host, such as skin or mucosal surfaces. Suitable compositions include semisolid compositions, viscous emulsions, sprays, washes, foams, depositories or other implanted depots comprising RIP. A nanoparticle carrier system provides an advantageous delivery vehicle for delivery of RIP compositions externally or internally, providing greater shelf life and in vivo stability than other colloidal delivery systems. Greater in vivo stability allows more sustained delivery and ultimately greater accumulation of RIP compositions in blood or tissues subject to bacterial infection. Nanoparticles comprising a RIP composition preferably are administered through oral or nasal routes, although the nanoparticles also may be administered parenterally or topically, depending on the desired distribution of the RIP composition. In one embodiment, the composition used to deliver RIP externally comprises nanoparticles containing RIP. In another embodiment, the nanoparticles are delivered in a reservoir, e.g., capsule or tablet, or coating of a device that is ingested or implanted into a host, providing sustained local delivery of a RIP composition.

According to first aspect of the invention, nanoparticles comprise a RIP composition, which comprises an RNAIII-inhibiting peptide and optionally additional active agents complementing or facilitating the antimicrobial effect of RIP, such as antibiotics or antimicrobial peptides. The composition may further comprise other pharmaceutically acceptable agents, such as agents that assist or delay adsorption of the composition by the host.

The RIP may comprise five contiguous amino acids of the sequence YX₂PX₁TNF, where X₁ is C, W, I or a modified amino acid, and X₂ is K or S; or amino acids having a sequence that differs from the sequence YX₂PX₁TNF by two substitutions or deletions, where X₁ is C, W, I or a modified amino acid, and X₂ is K or S. In one embodiment, the RIP does not consist of the sequence YSPX₁TNF, where X₁ is C, W, I or a modified amino acid. Alternatively, the RIP may comprise amino acids having a sequence that differs from the sequence YX₂PX₁TNF by one substitution or deletion, where X₁ is C, W, I or a modified amino acid, and X₂ is K or S. In various other embodiments, the RIP comprises the amino acid sequences YKPX₁TNF, where X₁ is C, W, I or a modified amino acid; the amino acid sequence IKKYX₂PX₁TNF, where X₁ is C, W, I or a modified amino acid and X₂ is K or S; or one of the sequences PCTNF, YKPITNF, or YKPWTNF. The RIP may be ten amino acids in length and may comprise about 0.1% to 50% by weight of the composition, or about 2% to 20% by weight of the composition.

According to a second aspect of the invention, a method of treating a disease associated with a bacterial infection comprises administering a composition comprising a RIP that is formulated in a nanoparticle carrier system. The method may be used to treat a systemic bacterial infection, or an infection localized to particular tissue, skin or region of the body. The infection may be associated with bacterial sepsis, cellulitis, keratitis, osteomyelitis, septic arthritis or mastitis, or the method may be used in the treatment of bacterial infection associated with biofilms, or in reducing the risk of a disease associated with biofilms. For example, the present composition may be used in a coating of a device inserted into an individual to reduce the risk that the implanted device will develop a biofilm. The nanoparticle carrier system is formulated appropriately for the method in which it is delivered to the individual in need thereof. For example, when nanoparticles comprising a RIP composition are injected or injected, they may be formulated in a liquid suspension comprising pharmacologically acceptable liquid dispersants. When applied to the skin or mucosal surfaces, the nanoparticles may be contained in a semisolid composition or viscous emulsion, e.g., a salve. Other suitable formulations include sprays, foams, washes, implants, depots, etc., depending on the nature of the infection being treated and the desired release kinetics. In one embodiment, the nanoparticles exhibit “burst-release kinetics,” which means substantial release from the nanoparticle carriers within 1, 2, 3, 7, or 24 hours after administration.

According to a third aspect of the invention, a RIP composition is delivered to an external surface of an individual, which may be skin or a mucosal membrane. Depending on the particular application, the RIP composition may be delivered in semisolid compositions, viscous emulsions, sprays, washes, foams, depositories or other implanted depots comprising RIP or the like, so that the formulation advantageously prolongs exposure of RIP to the desired surface.

The method further may be practiced on an individual at risk of having or suspected of having an infection caused by a bacteria, such as an individual who is suffering from burns, trauma, etc. Alternatively, the composition may be administered to treat an ongoing infection, delay the onset of symptoms of bacterial infection, or reduce the risk of developing an infection.

In one embodiment, the individual receiving the composition is infected or at risk of infection by bacteria in which RNAIII or TRAP plays a role in pathogenesis. In another embodiment, the infection or risk thereof is due to Gram-positive bacteria, such as Streptococcus spp, including S. aureus and S. epidermidis, or an antibiotic resistant strain thereof. In other embodiments, the pathogen may be Listeria spp, including L. innocua, and L. monoctogenes, Lactococcus spp, Enterococcus spp, Escherichia coli, Clostridium acetobtylicum, and Bacillus spp., including B. subtilus, B. anthracis, and B. cereus, or an antibiotic resistant strain thereof.

### BRIEF DESCRIPTION OF THE DRAWINGS

FIG. 1A depicts the regulation of bacterial virulence via TRAP and agr.

FIG. 1B depicts the role of RIP in decreasing bacterial virulence by decreasing TRAP phosphorylation.

FIG. 1C depicts the antagonistic effect of RAP, which increases TRAP phosphorylation.

FIG. 2 depicts typical results of a representative in vitro β-lactamase assay for testing activity of RIP compositions of the invention.

FIG. 3 depicts a representative outcome in a mouse sepsis/cellulitis model, where an intravenous or oral administration of a RIP composition protected the top mouse from S. aureus infection, but the bottom mouse was unprotected. Lesions caused by S. aureus infection are apparent on the bottom mouse.

FIG. 4 depicts a rat graft model system, which is representative of animal models useful for testing RIP compositions of the present invention.

### DETAILED DESCRIPTION

RIP compositions may be formulated to reduce toxicity, achieve sustained release, protect RIP from degradation and achieve improved therapeutic efficacy, similar to the advantages achieved when RIP compositions are formulated with liposomes. The use of nanoparticle carriers is particularly advantageous for oral or nasal administration of a composition comprising RIP.

RNAIII-Inhibiting Peptides of the Invention

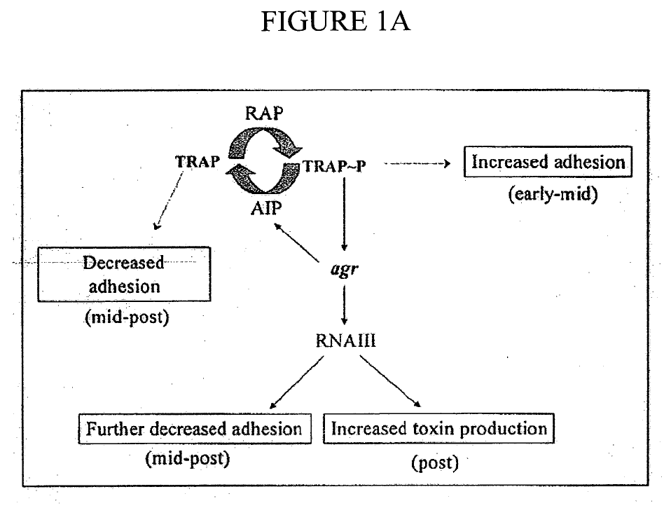

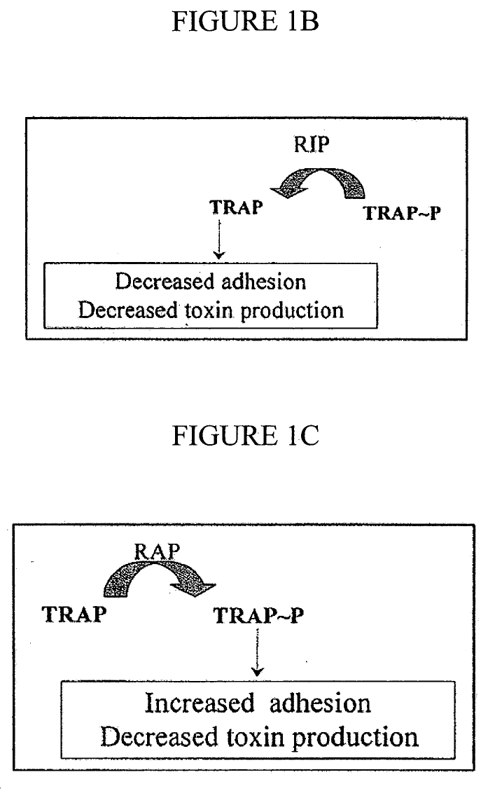

The quorum-sensing inhibitor RIP does not affect bacterial growth but reduces the pathogenic potential of the bacteria by interfering with the signal transduction that leads to production of exotoxins. RIP blocks toxin production by inhibiting the phosphorylation of its target molecule TRAP, which is an upstream activator of the agr locus. FIG. 1A depicts the role of TRAP phosphorylation in the downstream activation of the agr locus. As cells multiply, RAP accumulates in the extracellular milieu and promotes TRAP phosphorylation, leading to increased bacterial adhesion and agr activation in the mid-exponential stage of growth. Agr activation leads to the production of Autoinducing Peptide (AIP), which reduces TRAP phosphorylation but allows expression of RNAIII, which increases hemolysin and enterotoxin production. FIG. 1B depicts the role of RIP or RIP agonists, such as anti-RAP antibodies, in inhibiting TRAP phosphorylation, shifting the equilibrium to the non-phosphorylated, inactive form of the TRAP enzyme and blocking agr expression, thereby decreasing the adherence, biofilm formation, and toxin production of the bacteria. FIG. 1C depicts the effect of RAP in promoting TRAP phosphorylation, antagonizing the activity of RIP.

RIP comprises the general formula YX₂PX₁TNF, where X₁ is C, W, I or a modified amino acid and X₂ is K or S. Specific RIP sequences are disclosed in U.S. Pat. No. 6,291,431, application Ser. No. 10/358,448, filed Feb. 3, 2003, application Ser. No. 09/839,695, filed Apr. 19, 2001, and Gov et al., Peptides 22:1609-20 (2001), all of which are incorporated herein by reference. RIP sequences include polypeptides comprising the amino acid sequence KKYX₂PX₁TN, where X₁ is C, W, I or a modified amino acid and X₂ is K or S. RIP sequences also include polypeptides comprising YSPX₁TNF, where X₁ is C or W, and YKPITN. In one embodiment, the RIP comprising the general formula YX₂PX₁TNF above is further modified by one or two amino acid substitutions, deletions, and other modifications, provided the RIP exhibits activity.

The terms “protein,” “polypeptide,” or “peptide,” as used herein with reference to both RIP and antimicrobial peptides, include modified sequences (e.g., glycosylated, PEGylated, containing conservative amino acid substitutions, containing protective groups, including 5-oxoprolyl, amidation, D-amino acids, etc.). Amino acid substitutions include conservative substitutions, which are typically within the following groups: glycine, alanine; valine, isoleucine, leucine; aspartic acid, glutamic acid; asparagine, glutamine; serine, threonine; lysine, arginine; and phenylalanine, tyrosine.

Proteins, polypeptides and peptides of the invention can be purified or isolated. “Purified” refers to a compound that is substantially free, e.g., about 60% free, about 75% free, or about 90% free, from components that normally accompany the compound as found in its native state. An “isolated” compound is in an environment different from that in which the compound naturally occurs. Proteins, polypeptides and peptides of the invention may be naturally occurring or produced recombinantly or by chemical synthesis according to methods well known in the art.

RIP Compositions for External Application

RIP compositions may be formulated specifically for application to external surfaces of an individual, where “external surfaces” include the skin or mucosal surfaces. Preferred sites of administration include burns, wounds or other openings in the skin or mucosal surfaces that are at particular risk of infection. Suitable compositions for such applications include semisolid compositions, such as ointments, unctions, balms, creams or the like. Viscous emulsions may be useful, where the RIP is dissolved in one phase of a colloidal suspension. For example, RIP may be dissolved in an aqueous phase in a partly oil-based emulsion. Useful emulsions would have a sufficiently high viscosity to allow easy application while maintaining a high local concentration of RIP. Sprays or foams provide convenient compositions for delivering RIP to the skin or otherwise poorly accessible mucosal surfaces. For example, nasal sprays are useful for delivering RIP to nasal mucosal surfaces. Foams are particularly useful for delivering RIP to gums, e.g. in dental applications. Depositories or other implanted devices are particularly useful for sustained delivery of RIP to otherwise inaccessible mucosal surfaces, especially those such as the colon, which provide rapid absorption of delivered drugs into the blood. Mucosal surfaces useful for topical application of RIP compositions include mucous membranes of the conjunctiva, nasopharynx, oropharynx, vagina, colon, urethra, or urinary bladder, which are preferred when rapid adsorption is desired. Methods for manufacturing compositions having the desired general characteristics are known in the art, as are principles of pharmacodynamics, drug absorption, bioavailability, administration, distribution and excretion. See, e.g., Remington, “The Science and Practice of Pharmacy,” Gennaro, ed., 20^{th} ed., Lippincott Williams & Wilkins (2000), especially at Part 5, (“Pharmaceutical Manufacturing”); Goodman and Gilman's, “The Pharmacologic Basis of Therapeutics,” Hardman et al., eds., 10^{th} ed., McGraw-Hill (2001), especially at Chapters 1 and 2. Remington and Goodman and Gilman's are both incorporated herein by reference in their entirety.

When a RIP composition is delivered via the skin, oil or hydrators can be added to the composition to facilitate adsorption through the skin. The composition may contain any combination of other ingredients that would relieve pain and swelling or otherwise promote healing, e.g., antibiotics, analgesics, anti-inflammatory agents. See, e.g., Remington (2000), especially at Part 7 (“Pharmaceutical and Medicinal Agents”). Other useful ingredients, such as agents that control viscosity or color of the composition, are well known in the art. These compositions may be formulated with or without nanoparticles, which are now described in more detail.

Oral and Nasal Delivery of RIP Compositions Using Colloidal Nanoparticles

The bioavailability of peptide and protein drugs after oral administration generally is very low because of peptide instability in the gastrointestinal (GI) tract and low permeability of peptides through the intestinal mucosa; therefore, peptide drugs are usually injected to obtain therapeutic effects. Oral and nasal administration, however, are more convenient and more tolerated routes for drug delivery.

To facilitate oral delivery of RIP compositions, RIP compositions may be incorporated in nanoparticle carriers to increase the surface area available for bioadsorption, thereby improving the release of RIP in GI fluids. Nanoparticles also possess improved bioadhesion, increasing the residence time in the GI tract of RIP compositions formulated in nanoparticles. These advantages of nanoparticles as drug carriers, of course, apply equally to formulations designed for administration through other routes. When delivered orally, it is expected that a fraction of nanoparticles are adsorbed intact through the Peyer's patches in the gastrointestinal tract.

Preferred nanoparticles comprise biodegradable and biocompatible polymers. Useful nanoparticles include biodegradable poly(alkylcyanoacrylate) nanoparticles made by the procedure set forth in Vauthier et al., Adv. Drug Del. Rev. 55: 519-48 (2003), herein incorporated by reference. Oral adsorption also may be enhanced using poly(-lactide-glycolide) nanoparticles coated with chitosan, which is a mucoadhesive cationic polymer. The manufacture of such nanoparticles is described, for example, in Takeuchi et al., Adv. Drug Del. Rev. 47: 39-54 (2001), also incorporated herein by reference.

Nanoparticles are suitable for controlled release of RIP compositions administered through intratracheal (IT) and intravenous routes, as well. Nasal delivery of RIP compositions may be facilitated by use of the positively charged nanoparticles described in Somavarapu et al., J. Pharm. Pharmacol. 54 (Supp.): 131 (2002). Further, Poyner et al., J. Control. Rel. 35: 41-48 (1995) demonstrated the usefulness of nanoparticles for both IT and intravenous delivery in a comparison of liposomes and poly(lactic acid) (PLA) nanoparticles as delivery vehicles for tobramycin to treat a Pseudomonas aeruginosa infection in an animal model. Liposomal encapsulation was more efficient than nanosphere loading (85% vs. 30% respectively). Both carriers were of the same size and charge, with liposomes slightly more negatively charged. A faster release profile was observed for the lipid carrier, although the nanoparticles advantageously showed burst-release kinetics. When administered intravenously, liposomal tobramycin was more efficiently retained in the lung compared to free or nanoparticle encapsulated tobramycin. Both nanospheres and liposomes, however, maintained a major proportion of the drug in the lung following IT dosing. It is thus expected that both nanoparticle and liposomal carriers will be useful delivery systems for RIP compositions and that each may offer advantages for particular applications.

Nanoparticle Formulations

Nanoparticles typically comprise either a polymeric matrix (“nanospheres”) or a reservoir system comprising an oily core surrounded by a thin polymeric wall (“nanocapsules”), where the core comprises the RIP composition. Polymers suitable for the preparation of nanoparticles include poly(alkylcyanoacrylates), and polyesters such as poly(lactic acid) (PLA), poly(glycolic acid), poly(-caprolactone) and their copolymers.

The nanoparticle may alternately or additionally comprise a coating or moiety, such as an antibody or fragment thereof, to assist in cell targeting. Nanoparticle size and morphology may be altered, as well, to yield formulations with desired physicochemical characteristics, loading, and controlled release properties appropriate for a RIP composition. By modifying the formulation appropriately, it is possible to mediate a burst release of RIP for the rapid onset of its antibacterial effects.

1. Biodegradable Polymers

Nanoparticles may be fabricated using biodegradable polyesters, e.g., polymers of poly(lactic acid) (PLA) and copolymers that are manufactured with varying quantities of glycolic acid (PLGA). PLA is more hydrophobic in comparison to PLGA; therefore, PLA offers a relatively extended release profile. Similarly, the ratio of glycolic acid to lactic acid in the copolymerization process effects the degradative properties of the resultant copolymer. In one embodiment, low molecular weight (14 kDa) PLGA is copolymerized with a high (50%) glycolide content (PLGA 50:50). These particles will degrade comparatively rapidly due to the low molecular weight and high glycolide content of the PLGA used. It is expected that 90% of the RIP will be released within 30 days, and 90% of the polymer will be resorbed within 5 weeks. To obtain nanospheres with an intermediate or long degradation profile, the aforementioned formulation may comprise a higher molecular weight copolymer (e.g., 60-100 kDa), with or without a lower glycolide content (PLGA 65:35 or 75:25). In short, a comprehensive range of PLA and PLGA polymer molecular weight, lactic/glycolic acid ratios, and PLA-PLGA blends may be used to optimize loading and release profiles.

RIP compositions may be associated with the nanospheres either by encapsulation, adsorption onto the particle surface, or both. Depending on the particular molecules in the RIP composition, peptide loading efficiencies of up to 100% are expected when a 10% w/w loading level is attempted. From previous encapsulation studies, an increase in drug loading is expected to increase in particle size; therefore, high and low peptide loading formulations may be used with large (˜2000-5000 nm average diameter) and small (˜200-500 nm average diameter) particle sizes, respectively. Note that the larger size particles are considered “nanoparticles” for the purpose of the invention, even though their diameters may exceed a micron.

2. Double Emulsion Solvent Evaporation

Nanoparticles may be prepared using an emulsification and solvent evaporation process, or so-called double emulsion process, for example. Other procedures comprise adding additional polymers through covalent modification of existing nanoparticles. To form a primary emulsion, an internal aqueous phase that contains a stabilizing emulsifier and a RIP composition is added to an ice-cold organic phase. The stabilizing emulsifier may be 10% w/v polyvinyl alcohol (PVA), and the organic phase may comprise a polymer dissolved in dichloromethane (DCM). The polymer content is modified according to the particle size required.

In one embodiment, the primary emulsion is homogenized on ice for 3 minutes at high speed (16,000 RPM) using a high-speed homogenizer (Silverson). The primary emulsion then is added to a continuous phase consisting of 3% w/v PVA and homogenized for 6 minutes on ice to produce a secondary emulsion. The secondary emulsion is stirred at ˜400 RPM overnight in a fume hood to let the organic solvent evaporate. The particles are recovered by repeated (3×) high-speed centrifugation (˜20,000×g, depending on particle size) at 4° C. for 10 minutes and resuspended in double distilled water (ddH₂O). For optimal storage and stability, the particles are lyophilized.

3. Double Emulsion Solvent Diffusion

PLGA nanoparticles alternatively may be prepared using an emulsification-diffusion method. For example, 200 mg PLGA is dissolved in 10 ml of solvent, e.g., ethyl acetate and benzyl alcohol. The organic phases are added into 20 ml of an aqueous phase containing a stabilizer, e.g. poloxamer 188. After mutual saturation of organic and continuous phases, the mixture is emulsified for 7 min with a high-speed homogenizer (Silverson, United Kingdom) at 12,000 RPM. For full diffusion into the water phase, 500 ml of water are added to the O/W emulsion under moderate magnetic stirring, leading to the precipitation of the polymer as nanoparticles. In the case of drug-loaded nanoparticles, a predetermined amount of peptide first is dissolved in the organic solvent. A similar emulsion technique for making nanoparticles is described in Lamprecht et al., J. Pharmacol. Exp. Ther. 299: 775-81 (2001). The artisan skilled in the relevant art knows how to make various changes and modifications to this procedure.

4. Surface Modification of Nanoparticles

The surface of hydrophobic nanoparticles may be modified to minimize phagocytosis, allowing sustained systemic circulation of nanoparticles. Following intravenous administration, hydrophobic nanoparticles are rapidly cleared from systemic circulation by the mononuclear phagocytic system (MPS), resulting in rapid deposition of nanoparticles in the liver or spleen. When the liver, spleen or MPS itself are not targets of choice, various modifications of the nanoparticle surface are possible to minimize phagocytosis, including modification with poly(ethylene glycol) (PEG). PEG is a hydrophilic, nonionic polymer that exhibits excellent biocompatibility. PEG molecules, like other polymers, can be added to the nanoparticles by a number of different methods, including covalent bonding, blending during nanoparticle preparation, or surface adsorption. The presence of PEG on the nanoparticle surface serves other functions besides increasing residence time in systemic circulation. PEG has been shown to reduce protein and enzyme adsorption to the nanoparticle, retarding degradation of PLGA-based nanoparticles. The density and molecular weight of PEG on the surface can be adjusted to minimize protein adsorption. Poloxamer and poloxamines also have been shown to reduce nanoparticle capture by macrophages and increase nanoparticle residence time in systemic circulation. PLGA particles also may be coated with poloxamer 407 and poloxamine 908 to extend the half-life of the nanoparticles. Poly(ethylene glycol) can be introduced at the surface either (a) by adsorption of surfactants (e.g., poloxamer 188) or (b) as block or branched co-polymers, usually based on polyesters, such as poly(lactic acid) (PLA). Further, as mentioned above, cell targeting also may utilize molecules, e.g. epitope binding regions of antibodies, having specific affinity for moieties on the surface of targeted cells.

5. Physicochemical Characterization

The size distribution of nanoparticles influences their biodistribution and degradation kinetics. Nanoparticle size distribution, as well as surface and structural characteristics, may be visualized directly by transmission, scanning, or field-emission electron microscopy, where the particle size is 50 nm or higher. Electron microscopy may be complemented by fluorescent microscopy or confocal microscopy, using an entrapped fluorescently labeled marker peptide. Further, laser diffractometry (Mastersizer, Malvern, United Kingdom) or photon correlation spectroscopy (PCS, Malvern) may be used to determine the size distribution of nanoparticles 200 nm or more in diameter, and multi-angle PCS will be used for nanoparticle sizes in the 10 nm-5000 nm range.

As mentioned above, relative surface hydrophobicity also plays a role in nanoparticle pharmacokinetics. An adaptation of the Rose-Bengal assay, for example, may be used to determine hydrophobicity of nanoparticles. See Muller et al., Biochem. Soc'y Trans. 19: 502 (1991). In this method, the hydrophobic Rose-Bengal dye binds a quantity of particles relative to the strength of hydrophobic interactions between the dye and the nanoparticles. Unbound dye is quantified, and hydrophobicity is expressed as the percentage of bound dye mass. In this assay, an equal volume of 10 mg/ml Rose-Bengal dye typically is dissolved in ddH₂O and added to an aqueous suspension of nanoparticles. Following gentle agitation overnight at room temperature, the samples are centrifuged at 15,000×g for 30 min at room temperature. The supernatant is removed, and absorbance is read at 547 nm using a spectrophotometer.

Freeze-dried nanoparticle formulations can be desiccated at room temperature and ambient light conditions over two years without particle degradation, physicochemical changes, or loss of antigenicity. Formulations preferably are freeze-dried and sealed under vacuum or nitrogen to minimize degradation.

6. Peptide Loading and Release

To measure peptide loading, RIP-loaded nanoparticles may be quantitatively dissolved in methylene chloride and the peptide extracted into acetate buffer (pH 4, 0.1 M) by shaking the mixture for 1 h on a wrist action shaker. The aqueous buffer phase is separated by centrifugation, and extracted peptide is quantified by reverse phase-HPLC. The drug content may be expressed as % w/w of nanoparticles. To identify in vitro release profiles, 4 mg quantities of peptide-loaded particles (n=3 per time point) are incubated in 1 ml of release medium (0.01% v/v Tween 20, 0.1% NaN₃ in PBS) at 37° C. under gentle agitation at various time points: 1, 2, 3, 7, and 24 hours (the “burst release” period), 7 and 14 days, and 1 and 3 months. Upon collection, the particles will be centrifuged at 15,000×g for up to 45 minutes, depending on particle size at room temperature. Total peptide release at a time point can be quantified in the supernatants by HPLC.

7. Peptide Integrity

The integrity of peptides contained in nanoparticles is important when developing nanoparticles using various manufacturing methods. Typically, >95% peptide integrity can be maintained following formulation. In all assessments of peptide stability, HPLC can be used to evaluate the proportion of intact peptide. In addition, the peptide can be radiolabeled to show retention of association and dissociation of the peptide with the nanoparticles. Briefly, the peptide is incubated in a phosphate buffer solution containing ¹²⁵I bound onto beads (IODO-BEADS®). Tyrosine residues (and to a lesser extent histidine and tryptophan) attach to the iodine and remove it from the beads. The excess free radionuclide is removed from solution by gel filtration using a desalting column.

8. Organ Localization and Biodistribution of RIP Compositions

To determine the biodistribution of the encapsulated RIP composition, radiolabeled formulations may be administered to rats. Animals are sacrificed, the liver, spleen, lugs, intestine and blood are collected, and radioactivity of the tissues is monitored. Pharmacokinetics of both free and colloidal encapsulated peptide may be compared at several intervals after administration of drug. The concentration of peptide will be determined by HPLC as described above.

Assay Systems for Determining Activity of RIP and RIP Formulations

The mechanism through which RIP inhibits quorum-sensing mechanisms, as discussed above, involves inhibition of the phosphorylation of TRAP. There is evidence of the presence of TRAP and TRAP phosphorylation in S. epidermidis, indicating that there is a similar quorum sensing mechanisms both in S. aureus and in S. epidermidis and the potential for RIP to interfere with biofilm formation and infections caused by both species. In addition, there is evidence that TRAP is conserved among all staphylococcal strains and species; therefore, RIP should be effective against any type of Staphylococcus. Further, other infection-causing bacteria appear to have proteins with sequence similarity to TRAP, including Bacillus subtilus, Bacillus anthracis, Bacillus cereus, Listeria innocua, and Listeria monoctogenes. Moreover, RAP is an ortholog of the ribosomal protein L2, encoded by the rplB gene. See Korem et al., FEMS Microbiol. Lett. 223: 167-75 (2003), which is incorporated by reference herein with regard to its description of RAP orthologs encoded by the rplB gene. L2 is highly conserved among bacteria, including Streptococcus spp, Listeria spp, Lactococcus spp, Enterococcus spp, Escherichia coli, Clostridium acetobtylicum, and Bacillus spp. This finding indicates that treatment aimed at disturbing the function of RAP in S. aureus also will be effective in treating L2-synthesizing bacteria as well.

Preferred RNAIII-inhibiting peptides according to the invention directly or indirectly exhibit RNAIII inhibiting activity, which can be assayed using a number of routine screens. RIP inhibits Staphylococcus adherence and toxin production by interfering with the known function of a staphylococcal quorum-sensing system. As discussed above, RIP competes with RAP induction of TRAP phosphorylation, leading to the inhibition of TRAP phosphorylation. See Balaban et al., J. Biol. Chem. 276: 2658-67 (2001). This decreases cell adhesion, biofilm formation, and RNAIII synthesis and ultimately suppresses the virulence phenotype. See Balaban et al., Science 280: 438-40 (1998). For example, RIP inhibition of RNAIII production or TRAP phosphorylation can be assayed in vitro using the procedures described in Balaban et al., Peptides 21:1301-11 (2000), incorporated herein by reference in its entirety. The activity of the amide form of a synthetic RIP analogue YSPWTNF(-NH₂) can be demonstrated in a cellulitis model, using Smith Diffuse mice infected with S. aureus, in a septic arthritis model, testing mice against S. aureus LS-1, in a keratitis model, testing rabbits against S. aureus 8325-4, in an osteomyelitis model, testing rabbits against S. aureus MS, and in a mastitis model, testing cows against S. aureus Newbould 305, AE-1, and environmental infections. See Balaban et al., Peptides 21:1301-11 (2000) and TABLE 1. (The non-amidated form of synthetic RIP is inactive.) These findings demonstrate the range of RIP activities and screens available to assay RIP activity and further indicate that RIP prevents and suppresses staphylococcal infections.

TABLE 1

| | | S. aureus | Animals tested (n) | | % animals | |
|---|---|---|---|---|---|---|
| Infection | Model | strain | −RIP | +RIP | disease free | P |
| Osteomyelitis | Rabbit | MS | 7 | 8 | 58 | 0.02 |
| Sepsis | Mouse | LS-1 | 10 | 11 | 44 | 0.04 |
| Arthritis | Mouse | LS-1 | 10 | 10 | 60 | 0.006 |
| Keratitis | Rabbit | 8325-4 | 8 | 8 | 40 | 0.015 |
| Mastitis | Cow | Newbould/AE-1 | 6 | 7 | 70-100 | <0.05 |
| Cellulitis/sepsis | Mouse | Smith diffuse | 22 | 20 | Up to 100 | 0.02 |
| Graft injection | Rat | MRSA, MRSE, VISA, VISE, | >1000 | >1000 | Up to 100 | <0.05 |
| | | GISA, GISE, MSSA, MSSE | | | | |

The screening assay can be a binding assay, wherein one or more of the molecules may be joined to a label that provides a detectable signal. Purified RIP further may be used to determine a three-dimensional crystal structure, which can be used for modeling intermolecular interactions. Alternatively, a screening assay can determine the effect of a candidate RIP on RNAIII production and/or virulence factor production. For example, the effect of the candidate peptide on rnaiii transcription in Staphylococcus can be measured. Such screening assays can utilize recombinant host cells containing reporter gene systems such as CAT (chloramphenicol acetyltransferase), β-galactosidase, and the like, according to well-known procedures in the art. Alternatively, the screening assay can detect rnaiii or virulence factor transcription using hybridization techniques that also are well known in the art.

In vitro High Throughput Analysis of RIP Formulations

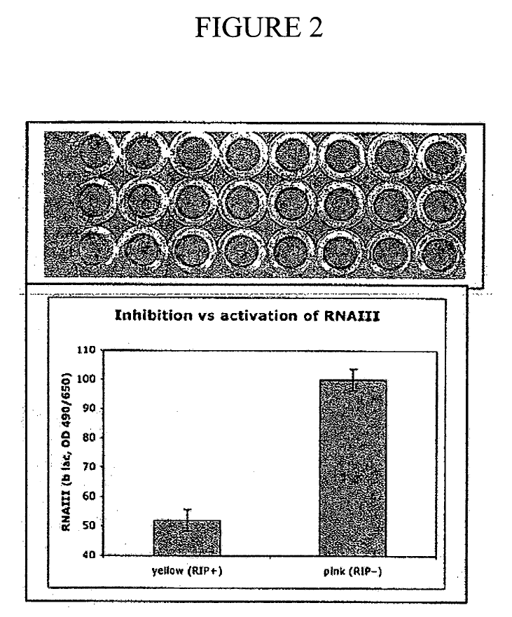

The following screening assay for RIP compositions exemplifies the types of assays that may be used to determine whether a particular RIP or RIP composition or formulation exhibits the desired level of biological activity. In this assay system, agr expression is tested in a high throughput assay using an RNAIII reporter gene assay, which is confirmed by Northern blotting. S. aureus cells in early exponential growth (about 2×10⁷ colony forming units (CFU)) containing the rnaiii::blaZ fusion construct are grown with increasing concentrations of the RIP formulations in 96 well plates at 37° C. with shaking for 2.5-5 hrs. In this assay, β-lactamase acts as a reporter gene for RNAIII. Bacterial viability is tested by determining O.D. 650 nm and further by plating to determine CFU. β-lactamase activity is measured by adding nitrocefin, a substrate for β-lactamase. Hydrolysis of nitrocefin by β-lactamase is indicated by a change in relative adsorption at 490 nm and 650 nm, where yellow color indicates no RNAIII synthesis, and pink color indicates RNAIII synthesis. Typical results of a β-lactamase reporter assay are shown in FIG. 2.

Formulations showing efficacy in the high throughput assay may be confirmed by Northern blotting. Bacteria are similarly grown with candidate RIP formulations. Cells are then collected by centrifugation, and total RNA is extracted and separated by agarose gel electrophoresis and Northern blotted. RNAIII is detected by hybridization to radio-labeled RNAIII-specific DNA produced by PCR, for example. Control formulations, containing random peptides typically are tested at 0-10 μg/10⁷ bacteria.

In vivo Analysis of RIP Formulations

Candidate peptides also can be assayed for activity in vivo, for example by screening for an effect on Staphylococcus virulence factor production in a non-human animal model. The candidate peptide is administered to an animal that has been infected with Staphylococcus or that has received an infectious dose of Staphylococcus in conjunction with the candidate peptide. The candidate peptide can be administered in any manner appropriate for a desired result. For example, the candidate peptide can be administered by injection intravenously, intramuscularly, subcutaneously, or directly into the tissue in which the desired affect is to be achieved, or the candidate can be delivered topically, orally, etc. The peptide can be used to coat a device that will then be implanted into the animal. The effect of the peptide can be monitored by any suitable method, such as assessing the number and size of Staphylococcus-associated lesions, microbiological evidence of infection, overall health, etc.

The selected animal model will vary with a number of factors known in the art, including the particular pathogenic strain of Staphylococcus or targeted disease against which candidate agents are to be screened. For example, when assessing the ability of the RIP formulation to suppress infections associated with toxin production, a mouse sepsis/cellulitis model is particularly useful. Balaban et al., Science 280: 438-40 (1998). This model is particularly preferred, for example, when the formulation comprises a RIP and a polycationic antimicrobial peptide that is capable of binding and neutralizing bacterial exotoxins and toxic cell wall components, which otherwise would induce an inflammatory response and toxic shock syndrome.

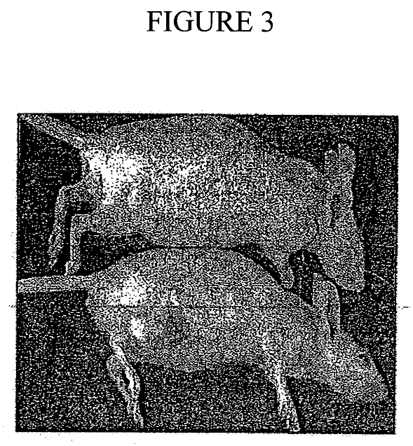

In the mouse sepsis cellulitis model, hairless immunocompetent mice (n=10) typically are challenged by a subcutaneous injection with 100 μL saline containing 5×10⁸ CFU S. aureus strain Smith diffuse together with cytodex beads. Formulated RIP is administered by intravenous administration or by oral gavage at ten times the i.v. dose. A typical i.v. dose will be <10 mg RIP/kg host body weight. Animals are observed for five days, and lesions are measured. It is expected that some of the animals will die of sepsis within the first 48 hrs due to the infection and others will develop lesions of various sizes. FIG. 3 shows typical lesions and the prevention of the same by a RIP formulation, where the bottom mouse is unprotected by a RIP formulation.

A rat graft model is especially useful to assess the ability of a formulation to suppress infections associated with biofilm formation. Giacometti et al., Antimicrob. Agents Chemother. 47: 1979-83 (2003); Cirioni et al., Circulation 108: 767-71 (2003); Balaban et al., J. Infect. Dis. 187: 625-30 (2003). This model is highly relevant to the clinical setting because it provides a time interval between bacterial challenge and biofilm infection, typically within 72 hours, allowing testing of the optimal route of administration and dose of the RIP formulation. This model provides a challenging test of RIP activity because biofilms are known to be extremely resistant to antibiotics.

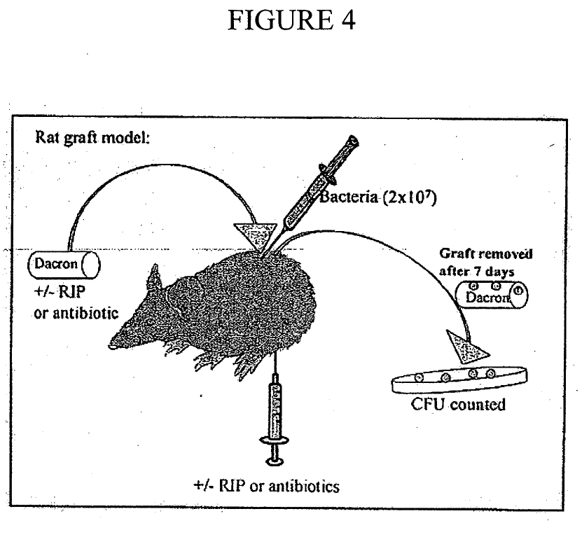

The typical steps in a rat graft model are shown in FIG. 4. Using this test, RIP was shown to reduce infection by four orders of magnitude when grafts were soaked with 20 μg/mL RIP for 20 minutes or when RIP was injected by an intraperitoneal route at 10 mg RIP/kg body weight. These results with the rat graft model will be repeated with the most promising formulations determined using the in vitro assays above, using higher or lower RIP concentrations than used with RIP alone. Formulation efficacy then can be compared to intraperitoneal RIP administration at doses known to be effective. RIP formulation can be administered daily before and/or after biofilm formation for 0-6 days after bacterial challenge.

In a typical experiment, Wistar adult male rats (n=10) are anesthetized, and a subcutaneous pocket is made on each side of the median line by a 1.5 cm incision. Sterile collagen-sealed double velour knitted polyethylene terephthalate (Dacron) grafts (1 cm²) (Albograft™, Italy) are soaked with saline, a random peptide having no RIP activity, RIP, or a nanoparticle formulation comprising RIP and implanted into the pockets. Pockets are closed with skin clips, and 2×10⁷ CFU/mL bacteria are inoculated onto the graft surface using a tuberculin syringe to create a subcutaneous fluid-filled pocket. The animals are returned to individual cages and examined daily. Animals receive an intravenous or oral administration of RIP or a RIP formulation 0-6 days after the graft infection. Free RIP is administered via an intraperitoneal route as a positive control. Grafts are explanted at 7 days following implantation and CFU are determined according to know procedures, e.g., Giacometti et al. (2003). The explanted grafts are placed in sterile tubes, washed in sterile saline solution, placed in tubes containing 10 mL of phosphate-buffered saline solution, and sonicated for 5 minutes to remove the adherent bacteria from the grafts. After sonication, grafts are microscopically checked to verify that all bacteria are removed. (No significant differences in cell viability (CFU/mL) were present upon testing the effect of sonication for up to 10 minutes on either antibiotic sensitive or antibiotic resistant bacteria.) Viable bacteria are quantified by culturing serial dilutions (0.1 mL) of the bacterial suspension on blood agar plates. All plates are incubated at 37° C. for 48 hours and evaluated for number of CFUs per plate. The limit of detection for this method is approximately 10 CFU/mL.

Methods of Treating

The term “treatment” or “treating” means any therapeutic intervention in an individual animal, e.g., a mammal, preferably a human. Treatment includes (i) “prevention,” causing the clinical symptoms not to develop, e.g., preventing infection from occurring and/or developing to a harmful state; (ii) “inhibition,” arresting the development of clinical symptoms, e.g., stopping an ongoing infection so that the infection is eliminated completely or to the degree that it is no longer harmful; and (iii) “relief,” causing the regression of clinical symptoms, e.g., causing a relief of fever and/or inflammation caused by an infection. Treatment may comprise the prevention, inhibition, or relief of biofilm formation. Administration to an individual “at risk” of having a bacterial infection means that the individual has not necessarily been diagnosed with a bacterial infection, but the individual's circumstances place the individual at higher than normal risk for infection of infection, e.g., the individual is a burn victim. Administration to an individual “suspected” of having a bacterial infection means the individual is showing some initial signs of infection, e.g., elevated fever, but a diagnosis has not yet been made or confirmed.

The term “effective amount” means a dosage sufficient to provide treatment. The quantities of active ingredients necessary for effective therapy will depend on many different factors, including means of administration, target site, physiological state of the patient, and other medicaments administered; therefore, treatment dosages should be titrated to optimize safety and efficacy. Typically, dosages used in vitro may provide useful guidance in the amounts useful for in vivo administration of the active ingredients. Animal testing of effective doses for treatment of particular disorders will provide further predictive indication of human dosage. The concentration of the active ingredients in the pharmaceutical formulations typically vary from less than about 0.1%, usually at or at least about 2% to as much as 20% to 50% or more by weight, and will be selected primarily by fluid volumes, viscosities, etc., in accordance with the particular mode of administration selected. Various appropriate considerations are described, for example, in Goodman and Gilman's, “The Pharmacological Basis of Therapeutics,” Hardman et al., eds., 10^{th} ed., McGraw-Hill, (2001) and “Remington: The Science and Practice of Pharmacy,” University of the Sciences in Philadelphia, 21^{st} ed., Mack Publishing Co., Easton Pa. (2005), both of which are herein incorporated by reference in their entirety. Methods for administration are discussed therein, including administration by oral, intravenous, intraperitoneal, intramuscular, transdermal, nasal, topical, and iontophoretic routes, and the like.

For the purpose of the invention, a “RIP composition” comprises an RNAIII-inhibiting peptide and possibly other pharmacologically active agents. Suitable active agents include antibiotics and antimicrobial peptides. Useful antibiotics include, but are not limited to, an amino-glycoside, (e.g., gentamycin), a beta-lactam (e.g., penicillin), or a cephalosporin. Useful antimicrobial peptides are described further below. Active agents may be administered to the individual in the same composition as the RIP or in a separate formulation at or around the same time as the RIP composition is administered. For example, the present method comprises oral co-administration of separate formulations of a RIP composition and an antibiotic. Administration of the RIP and antibiotic may occur within about 48 hours and preferably within about 2-8 hours, and most preferably, substantially concurrently.

The present composition is useful in reducing the overall pathology or delaying the onset of disease symptoms in various diseases caused by bacterial infection, including bacterial sepsis, bacterial-induced systemic inflammatory syndrome (SIRS), cellulitis, keratitis, osteomyelitis, septic arthritis, mastitis, skin infections, pneumonia, endocarditis, meningitis, post-operative wound infections, device-associated infections, periodontal infections and toxic shock syndrome.

Nanoparticle Compositions Comprising RIP

Nanoparticle formulations according to the invention comprise RIP compositions, as set forth above. RIP compositions comprise a RIP peptide in an amount effective to treat or reduce the risk of bacterial infection, and may additionally comprise other active ingredients that help promote the antibacterial activity of RIP. For example, a RIP composition may comprise an antimicrobial peptide or a conventional antibiotic. The composition also may comprise inactive ingredients, which may be added to the composition to provide desirable color, taste, stability, buffering capacity, dispersion or other features. These ingredients include iron oxide, silica gel, sodium lauryl sulfate, titanium dioxide, edible white ink, and the like.

The nanoparticle carriers themselves may be administered with or without additional agents. For example, if nanoparticles are administered orally or as a depot, they may be entrained in a liquid or contained within a capsule. Nanoparticles also may be contained in foams, salves or the like for the purpose of administration, for example. Conventional nontoxic solid carriers may be used in conjunction with nanoparticles, such as pharmaceutical grades of mannitol, lactose, starch, magnesium stearate, sodium saccharin, talcum, cellulose, glucose, sucrose, magnesium carbonate, and the like. For oral administration, a pharmaceutically acceptable nontoxic composition is formed by incorporating any of the normally employed excipients, such as those carriers previously listed, and generally 10-95%, and more preferably 25%-75 %, of a RIP.

The concentration of therapeutically active compound in the formulation may be varied to provide the optimum therapeutic response. For example, several divided doses may be administered daily or the dose may be proportionally reduced as indicated by the therapeutic situation. Human dosage levels for treating infections are known and generally include a daily dose from about 0.1 to 500 mg/kg of body weight per day, preferably about 6 to 200 mg/kg, and most preferably about 12 to 100 mg/kg. The amount of formulation administered will, of course, be dependent on the subject and the severity of the affliction, the manner and schedule of administration and the judgment of the prescribing physician. When administered intravenously, for example, serum concentrations can be maintained at levels sufficient to treat infection in less than 10 days, although an advantage offered by the present invention is the ability to extend treatment for longer than 10 days at relatively low levels of the RIP composition because of the decreased likelihood that bacteria will develop resistance to the present composition over a long course of treatment.

Pharmaceutical grade organic or inorganic carriers or diluents can be used to make up compositions containing the therapeutically active compounds. Diluents known to the art include aqueous media, vegetable and animal oils and fats. Stabilizing agents, wetting and emulsifying agents, salts for varying the osmotic pressure or buffers for securing an adequate pH value, and skin penetration enhancers can be used as auxiliary agents. The compositions may include other pharmaceutical excipients, carriers, etc. Suitable excipients are, for example, water, saline, dextrose, glycerol, ethanol or the like. Methods of preparing pharmaceutical compositions are well known to those skilled in the art. See, for example, “Remington: The Science and Practice of Pharmacy,” University of the Sciences in Philadelphia, 21^{st} ed., Mack Publishing Co., Easton Pa. (2005).

Treatment of Biofilm-Related Infections

Bacteria that attach to surfaces aggregate in a hydrated polymeric matrix of their own synthesis to form biofilms. Formation of these sessile communities and their inherent resistance to antimicrobial agents are at the root of many persistent and chronic bacterial infections. See Costerton et al., Science 284: 1318-22 (1999). Biofilms develop preferentially on inert surfaces or on dead tissue and occur commonly on medical devices and fragments of dead tissue, such as sequestra of dead bone. Biofilms also can form on living tissues, as in the case of endocarditis. Biofilms grow slowly, in one or more locations, and biofilm infections are often slow to produce overt symptoms. Sessile bacterial cells release antigens and stimulate the production of antibodies, but the antibodies generally are ineffective against biofilms and may cause immune complex damage to surrounding tissues. Even in individuals with excellent cellular and humoral immune reactions, biofilm infections are rarely resolved by the host defense mechanisms. Antibiotic therapy typically reverses the symptoms caused by planktonic cells released from the biofilm, but fails to kill the biofilm. For this reason biofilm infections typically show recurring symptoms after cycles of antibiotic therapy, until the sessile population is surgically removed from the body. It is therefore preferable to prevent biofilm formation, rather than to try to eradicate biofilms once they have formed.

The composition and method of the present invention are useful treating bacterial infection associated with biofilms, or in reducing the risk of a disease associated with biofilms. For example, nanoparticles comprising a RIP composition may be used to coat devices that are inserted into an individual, e.g., a surgical device, catheter, prosthetic or other implant, to reduce the risk that the implanted device will develop a biofilm. Alternatively, the nanoparticles may be implanted to provide a high, localized concentration of the composition in the treatment of a localized infection. In this embodiment, the nanoparticles are formulated as a depot capable of sustained release. TABLE 2 below provides a partial list of nosocomial infections associated with biofilms, for which the present nanoparticle formulations and associated methods are expected to be useful.

TABLE 2

| Medical device or device- | Bacterial species typically responsible |
|---|---|
| associated disease | for associated biofilms |
| Sutures | S. aureus and S. epidermidis |
| Exit sites | S. aureus and S. epidermidis |
| Arteriovenous shunts | S. aureus and S. epidermidis |
| Schleral buckles | Gram-positive cocci |
| Contact lens | P. aeruginosa and |
| | Gram-positive cocci |
| Urinary catheter cystitis | E. coli and other |
| | Gram-negative rods |
| Peritoneal dialysis (CAPD) | Staphylococcus; |
| peritonitis | various bacteria and fungi |
| Endotracheal tubes | A variety of bacteria and fungi |
| Hickman catheters | S. epidermidis and C. albicans |
| ICU pneumonia | Gram-negative rods |
| Central venous catheters | S. epidermidis and others |
| Mechanical heart valves | S. aureus and S. epidermidis |
| Vascular grafts | Gram-positive cocci |
| Orthopedic devices | S. aureus and S. epidermidis |
| Penile prostheses | S. aureus and S. epidermidis |

Antimicrobial Peptides

As described above, the RIP formulations according to the present invention may comprise an antimicrobial peptide. Genetically encoded antimicrobial peptides are an important component of the innate immune response in most multi-cellular organisms that represents a first line of host defense against an array of microorganisms. Antimicrobial peptides have a broad spectrum of activities, killing or neutralizing both gram-negative and gram-positive bacteria, including antibiotic-resistant strains. See Hancock, Lancet Infect. Dis. 1: 156-64 (2001). Wang, University of Nebraska Medical Center, Antimicrobial Peptide Database, at http://aps.unmc.edu/AP/main.php (last modified Mar. 5, 2005), which is incorporated herein by reference in its entirety, provides a database of about 500 antimicrobial peptides with antibacterial activity that potentially are useful for the present invention. Antimicrobial peptides usually are made up of between 12 and 50 amino acid residues and are polycationic. Usually about 50% of their amino acids are hydrophobic, and they are generally amphipathic, where their primary amino acid sequence comprises alternating hydrophobic and polar residues. Antimicrobial peptides fit into one of four structural categories: (i) β-sheet structures that are stabilized by multiple disulfide bonds (e.g., human defensin-1), (ii) covalently stabilized loop structures (e.g., bactenecin), (iii) tryptophan (Trp)-rich, extended helical peptides (e.g., indolicidin), and (iv) amphipathic α-helices (e.g., the magainins and cecropins). See Hwang et al., Biochem. Cell Biol. 76: 235-46 (1998); Stark et al., Antimicrob. Agents Chemother 46: 3585-90 (2002).

Vaccines and Antibodies

Being a small peptide, RIP generally is non-immunogenic; however, the nanoparticle formulations, particularly those capable of sustained release, may be used to provoke an immune response, especially when used in combination with adjuvants and the like. An immune response to RIP advantageously antagonizes the activity of RAP, helping protect an individual from infection. See Gov et al. (2001). Accordingly, in one embodiment of the invention, RIP formulations are administered to an individual to provoke an immune response to RIP, creating antibodies that protect an individual from infection by antagonizing RAP function. Methods of subsequently creating monoclonal antibodies from antibody-producing B cells are well-known in the art, as are methods of analyzing antibodies and structurally manipulating antibodies through recombinant engineering. These and other methods relating to antibodies are described in application Ser. No. 10/358,448, filed Feb. 3, 2003; Ser. No. 09/839,695, filed Apr. 19, 2001; and Ser. No. 09/054,331, filed Apr. 2, 1998, now U.S. Pat. No. 6,291,431, each of which is incorporated herein by reference in their entirety.

All publications and patents mentioned herein are incorporated herein by reference to disclose and describe the specific methods and/or materials in connection with which the publications and patents are cited. The publications and patents discussed herein are provided solely for their disclosure prior to the filing date of the present application. Nothing herein is to be construed as an admission that the present invention is not entitled to antedate such publication or patent by virtue of prior invention. Further, the dates of publication or issuance provided may be different from the actual dates that may need to be independently confirmed.

While the foregoing specification teaches the principles of the present invention, with examples provided for the purpose of illustration, it will be appreciated by one skilled in the art from reading this disclosure that various changes in form and detail can be made without departing from the true scope of the invention.

What is claimed is:

1. A pharmaceutical composition comprising a polymeric nanoparticle comprising an RNAIII-inhibiting peptide (RIP).

2. The pharmaceutical composition of claim 1, wherein the RIP comprises:
(a) five contiguous amino acids of the sequence YX₂PX₁TNF, where X₁ is C, W, I or a modified amino acid, and X₂ is K or S; or
(b) amino acids having a sequence that differs from the sequence YX₂PX₁TNF by two substitutions or deletions, where X₁ is C, W, I or a modified amino acid, and X₂ is K or S.

3. The pharmaceutical composition of claim 2, where the RIP does not consist of the sequence YSPX₁TNF, where X₁ is C, W, I or a modified amino acid.

4. The pharmaceutical composition of claim 2, where the RIP comprises amino acids having a sequence that differs from the sequence YX₂PX₁TNF by one substitution or deletion, where X₁ is C, W, I or a modified amino acid, and X₂ is K or S.

5. The pharmaceutical composition of claim 2, where the RIP comprises the amino acid sequence YKPX₁TNF, where X₁ is C, W, I or a modified amino acid.

6. The pharmaceutical composition of claim 2, where X₂ in the RIP sequence is K.

7. The pharmaceutical composition of claim 2, where the RIP comprises the amino acid sequence IKKYX₂PX₁TNF, where X₁ is C, W, I or a modified amino acid, and X₂ is Kor S.

8. The pharmaceutical composition of claim 2, where the RIP comprises the sequence PCTNF, YKPITNF, or YKPWTNF.

9. The pharmaceutical composition of claim 2, where the RIP is ten amino acids in length.

10. The pharmaceutical composition of claim 1, where the nanoparticle fuirther comprises an antibiotic.

11. The pharmaceutical composition of claim 10, where the antibiotic is an amino-glycoside or a beta-lactam.

12. The pharmaceutical composition of claim 1, where the nanoparticle further comprises an antimicrobial peptide.

13. The pharmaceutical composition of claim 1, where the nanoparticle comprises biodegradable polymers.

14. The pharmaceutical composition of claim 1, where the nanoparticle has an average diameter of about 10 to 5000 nm.

15. The pharmaceutical composition of claim 13, where the nanoparticle has an average diameter of about 2000-5000 nm.

16. The pharmaceutical composition of claim 13, where the nanoparticle has an average diameter of about 200 to 500 nm.

17. The pharmaceutical composition of claim 1, where the nanoparticle is positively charged.

18. The pharmaceutical composition of claim 1, where the nanoparticle comprises poly(alkylcyanoacrylate), poly(lactide-glycolide), poly(lactic acid), poly(glycolic acid), or poly(caprolactone) polymers.

19. The pharmaceutical composition of claim 18, where the nanoparticle comprises poly(lactic acid) (PLA) to glycolic acid ratio of about 50:50.

20. The pharmaceutical composition of claim 18, where the nanoparticle comprises poly(lactic acid) (PLA) to glycolic acid ratio of about 65:35 to about 75:25.

21. The pharmaceutical composition of claim 1, where the nanoparticle exhibits burst-release kinetics.

22. The pharmaceutical composition of claim 1, where the nanoparticle surface comprises poly(ethylene glycol), a poloxamer, or a poloxamine.

23. The pharmaceutical composition of claim 1, where the nanoparticle surface comprises a molecule having a specific affinity for a moiety on a surface of a targeted cell.

24. The pharmaceutical composition of claim 1, further comprising an adjuvant.

25. The pharmaceutical composition of claim 1, where the nanoparticle is a nanosphere.

26. The pharmaceutical composition of claim 1, where the nanoparticle is a nanocapsule.

27. A method of making a pharmaceutical composition comprising a polymeric nanoparticle comprising an RNAIII-inhibiting peptide (RIP).

28. The method of claim 27, where method comprises homogenizing an aqueous phase comprising the RIP and an organic phase comprising the polymer to create an emulsion.

29. The method of claim 27, further comprising solvent evaporation or solvent diffusion.

30. A method of treating or reducing the risk of a bacterial infection in an individual, comprising administering a polymeric nanoparticle comprising an RNAIII-inhibiting peptide (RIP) to an amount effective to treat or reduce the risk of bacterial infection in the individual.

31. The method of claim 30, where the nanoparticles are administered by an oral, intravenous, intraperitoneal, intramuscular, transdermal, nasal, topical, or iontophoretic route.

32. The method of claim 30, where the bacterial infection is related to bacterial sepsis, bacterial-induced systemic inflammatory syndrome (SIRS), cellulitis, keratitis, osteomyelitis, septic arthritis, mastitis, skin infections, pneumonia, endocarditis, meningitis, post-operative wound infections, device-associated infections, periodontal infections, or toxic shock syndrome.

33. The method of claim 30, where the bacterial infection is related to a biofilm.

34. A pharmaceutical composition comprising an RNAIII-inhibiting peptide (RIP) in an amount effective to treat or reduce the risk of a bacterial infection in which RNAIII plays a role when the pharmaceutical composition is delivered to the skin or mucosal surface of a mammalian individual.

35. The pharmaceutical composition of claim 34, where the composition is formulated as a semisolid composition, viscous emulsion, spray, wash, foam, depository or depot.

36. The pharmaceutical composition of claim 34, where the RIP is contained in polymeric nanoparticles.

37. The pharmaceutical composition of claim 35, where the nanoparticles are biodegradable.

38. The pharmaceutical composition of claim 34, where the composition further comprises an oil, skin hydrator, antibiotic, analgesic, or anti-inflammatory agent.
